(19) United States
(12) Patent Application Publication
(10) Pub. No.: US 2025/0017592 A1
(43) Pub. Date: Jan. 16, 2025

(54) OCCLUSIVE IMPLANT SYSTEM

(71) Applicants: BOSTON SCIENTIFIC SCIMED, INC., MAPLE GROVE, MN (US); BOSTON SCIENTIFIC MEDICAL DEVICE LIMITED, Galway (IE)

(72) Inventors: Ryan Robert Davis, Plymouth, MN (US); Peeyush Tomar, Muzaffarnagar (IN); Carly Rogers, Eden Prairie, MN (US); Matthew D. Himes, Ham Lake, MN (US); Aman Kumar Verma, Lakhisarai (IN)

(73) Assignees: BOSTON SCIENTIFIC SCIMED, INC., MAPLE GROVE, MN (US); BOSTON SCIENTIFIC MEDICAL DEVICE LIMITED, Galway (IE)

(21) Appl. No.: 18/770,224

(22) Filed: Jul. 11, 2024

Related U.S. Application Data
(60) Provisional application No. 63/526,298, filed on Jul. 12, 2023.

(51) Int. Cl. A61B 17/12; A61B 17/00

(52) U.S. Cl. CPC A61B 17/12122; A61B 17/00234; A61B 17/12031; A61B 17/12172; A61B 2017/00238; A61B 2017/00292; A61B 2017/00477; A61B 2017/1205; A61B 2217/007

(57) ABSTRACT

A delivery system for an occlusive implant includes a delivery sheath assembly and a hub assembly. The delivery sheath assembly includes a delivery sheath defining a delivery lumen adapted to accommodate an occlusive implant releasably secured to a core wire. A proximal fitting is secured to the delivery sheath. The hub assembly includes a hub body, a distal hub secured relative to the hub body and adapted to engage the proximal fitting of the delivery sheath assembly, a proximal hub secured to the hub body, and a side port coupled to hub body and adapted for flushing an interior of the delivery system. The delivery system includes one or more features that are adapted to limit relative rotation between the delivery sheath assembly and the hub assembly when the core wire is rotationally locked to the proximal hub.

### CROSS REFERENCE TO RELATED APPLICATIONS

This application claims the benefit of priority of U.S. Provisional Application No. 63/526,298 filed Jul. 12, 2023, the entire disclosure of which is hereby incorporated by reference.

### TECHNICAL FIELD

The disclosure relates generally to medical devices and more particularly to medical devices that are adapted for use in percutaneous medical procedures including implantation into the left atrial appendage (LAA) of a heart.

### BACKGROUND

The left atrial appendage is a small organ attached to the left atrium of the heart. During normal heart function, as the left atrium constricts and forces blood into the left ventricle, the left atrial appendage constricts and forces blood into the left atrium. The ability of the left atrial appendage to contract assists with improved filling of the left ventricle, thereby playing a role in maintaining cardiac output. However, in patients suffering from atrial fibrillation, the left atrial appendage may not properly contract or empty, causing stagnant blood to pool within its interior, which can lead to the undesirable formation of thrombi within the left atrial appendage.

Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the left atrial appendage. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a delivery system for an occlusive implant. The delivery system includes a delivery sheath assembly and a hub assembly that is adapted to be coupled with the delivery sheath assembly. The delivery sheath assembly includes a delivery sheath extending from a proximal end to a distal end, the delivery sheath defining a delivery lumen extending therethrough, the delivery lumen adapted to accommodate an occlusive implant releasably secured to a core wire, and a proximal fitting secured to the proximal end of the delivery sheath. The hub assembly includes a hub body, a distal hub secured relative to the hub body and adapted to engage the proximal fitting of the delivery sheath assembly, a proximal hub secured to the hub body, the proximal hub adapted to accommodate the core wire extending through the proximal hub, and a side port coupled to hub body and adapted for flushing an interior of the delivery system. The delivery system includes one or more features that are adapted to limit relative rotation between the delivery sheath assembly and the hub assembly when the core wire is rotationally locked to the proximal hub.

Alternatively or additionally, the proximal fitting may include a Luer fitting.

Alternatively or additionally, the proximal fitting and/or the distal hub may include one or more features that are adapted to limit relative rotation between the delivery sheath assembly and the hub assembly.

Alternatively or additionally, the distal hub may include a Luer adaptor.

Alternatively or additionally, the Luer adaptor may include a first protruding feature and a distal region of the hub body may include a second protruding feature adapted to engage the first protruding feature as a result of relative rotation between the Luer adaptor and the hub body. Engagement between the first protruding feature and the second protruding feature may limit relative rotation between the Luer adaptor and the hub body to less than 360 degrees.

Alternatively or additionally, engagement between the first protruding feature and the second protruding feature may limit relative rotation between the Luer adaptor and the hub body to about 330 degrees.

Alternatively or additionally, the hub assembly may further include a locking member moveable between an engaged position providing a rotation lock between the delivery sheath assembly and the hub assembly and a disengaged position permitting rotation between the delivery sheath assembly and the hub assembly. A spring may extend between an interior stop within the locking member and a backing plate molded into the hub body and may bias the stopper into the engaged position.

Alternatively or additionally, the Luer adaptor may include a proximal engagement feature and the locking member may include a distal engagement feature that is complementary to the proximal engagement feature. The distal engagement feature engages the proximal engagement feature when the locking member is in the engaged position. The distal engagement feature does not engage the proximal engagement feature when the locking member is in the disengaged position.

Alternatively or additionally, the hub body may further include a snap lock member extending radially outwardly from the hub body, and the locking member may be adapted to releasably engage the snap lock member when the stopper is in the disengaged position.

Alternatively or additionally, the Luer adaptor may include a narrowed proximal region including one or more longitudinally extending slots, and the hub body may include a main hub body section including a narrowed distal region, the narrowed distal region including one or more longitudinally extending tabs complementary to the one or more longitudinally extending slots, and a side port body section defining a longitudinal lumen extending therethrough.

Alternatively or additionally, the narrowed proximal region may extend proximally into the longitudinal lumen and the narrowed distal region may extend distally into the longitudinal lumen such that the one or more longitudinally extending tabs engage the one or more longitudinally extending slots to prevent relative rotation between the main hub body section and the Luer adaptor. The side port body section is allowed to rotate relative to the main hub body section and the Luer adaptor.

Another example may be found in a delivery system for an occlusive implant. The delivery system includes a delivery sheath defining a delivery lumen extending therethrough that is adapted to accommodate an occlusive implant releasably secured to a core wire, a Luer fitting secured to a proximal end of the delivery sheath, a hub body, a Luer adaptor secured relative to the hub body and adapted to engage the Luer fitting, and a proximal hub secured to the hub body, the proximal hub adapted to accommodate the core wire extending through the proximal hub. The Luer adaptor and/or the distal hub includes one or more features that are adapted to limit relative rotation between the Luer adaptor and the Luer fitting.

Alternatively or additionally, the Luer adaptor may include a first protruding feature and a distal region of the hub body may include a second protruding feature adapted to engage the first protruding feature as a result of relative rotation between the Luer adaptor and the hub body. Engagement between the first protruding feature and the second protruding feature may limit relative rotation between the Luer adaptor and the hub body to less than 360 degrees.

Alternatively or additionally, engagement between the first protruding feature and the second protruding feature may limit relative rotation between the Luer adaptor and the hub body to about 330 degrees.

Alternatively or additionally, the delivery system may further include a locking member moveable between an engaged position providing a rotation lock between the Luer fitting and the Luer adaptor and a disengaged position permitting rotation between the Luer fitting and the Luer adaptor. A spring extends between an interior stop within the locking member and a backing plate molded into the hub body and biases the stopper into the engaged position.

Alternatively or additionally, the Luer adaptor may include a proximal engagement feature and the locking member may include a distal engagement feature that is complementary to the proximal engagement feature. The distal engagement feature engages the proximal engagement feature when the locking member is in the engaged position. The distal engagement feature does not engage the proximal engagement feature when the locking member is in the disengaged position.

Alternatively or additionally, the hub body may further include a snap lock member extending radially outwardly from the hub body. The locking member may be adapted to releasably engage the snap lock member when the stopper is in the disengaged position.

Alternatively or additionally, the Luer adaptor may include a narrowed proximal region including one or more longitudinally extending slots. The hub body may include a main hub body section including a narrowed distal region, the narrowed distal region including one or more longitudinally extending tabs complementary to the one or more longitudinally extending slots, and a side port body section defining a longitudinal lumen extending therethrough.

Alternatively or additionally, the narrowed proximal region may extend proximally into the longitudinal lumen and the narrowed distal region may extend distally into the longitudinal lumen such that the one or more longitudinally extending tabs engage the one or more longitudinally extending slots to prevent relative rotation between the main hub body section and the Luer adaptor. The side port body section is allowed to rotate relative to the main hub body section and the Luer adaptor.

Another example may be found in a delivery system for an occlusive implant. The delivery system includes a delivery sheath defining a delivery lumen extending therethrough, an occlusive implant releasably secured to a core wire, the occlusive implant and the core wire disposed within the delivery lumen, a Luer fitting secured to a proximal end of the delivery sheath, a hub body, and a Luer adaptor secured relative to the hub body and adapted to engage the Luer fitting. The Luer adaptor and the distal hub each include one or more features that are adapted to limit relative rotation between the Luer adaptor and the Luer fitting.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of an illustrative delivery system for an occlusive device, with the occlusive device shown in a delivery configuration;

FIG. 2 is a side view of the illustrative delivery system for an occlusive device of FIG. 1, with the occlusive device shown in a deployed configuration;

FIG. 3 is a perspective view of an illustrative hub assembly that may be used as part of the delivery system for an occlusive device shown in FIGS. 1 and 2;

FIG. 4 is an exploded perspective view of the illustrative hub assembly of FIG. 3;

FIG. 5 is a perspective view of a hub body forming part of the illustrative hub assembly of FIG. 3, showing a first protruding feature;

FIG. 6 is a perspective view of a proximal end of a Luer adaptor forming part of the illustrative hub assembly of FIG. 3, showing a second protruding feature;

FIG. 7 is a perspective view of a distal end of the Luer adaptor of FIG. 6;

FIG. 8 is a perspective view of an illustrative hub assembly that may be used as part of the delivery system for an occlusive device shown in FIGS. 1 and 2, shown in a locked position;

FIG. 9 is a perspective view of the illustrative hub assembly of FIG. 8, shown in an unlocked position;

FIG. 10 is a partial cutaway view of the illustrative hub assembly of FIG. 9;

FIG. 11 is a perspective view of a hub body forming part of the illustrative hub assembly of FIGS. 8 and 9;

FIG. 12 is a perspective view of a proximal end of a Luer fitting forming part of the illustrative hub assembly of FIGS. 8 and 9, showing a proximal engagement feature;

FIG. 13 is a perspective view of a distal end of a locking member forming part of the illustrative hub assembly of FIGS. 8 and 9, showing a distal engagement feature;

FIG. 14 is a perspective view of an illustrative hub assembly that may be used as part of the delivery system for an occlusive device shown in FIGS. 1 and 2;

FIG. 15 is an exploded perspective view of the illustrative hub assembly of FIG. 14;

FIG. 16 is a perspective view of the illustrative hub assembly of FIG. 14, with a side port body section of the hub body removed to show an interaction between a Luer adaptor and a main body section of the hub body;

FIG. 17 is a perspective view of an illustrative hub assembly that may be used as part of the delivery system for an occlusive device shown in FIGS. 1 and 2;

FIG. 18 is an exploded perspective view of the illustrative hub assembly of FIG. 17, showing a first locking feature;

FIG. 19 is a perspective view of a component of the illustrative hub assembly of FIG. 17, showing a complementary second locking feature; and

FIG. 20 is a perspective view of a component of the illustrative hub assembly of FIG. 17.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

In some instances, elements that are separately described may be made separately and then joined together. In some instances, elements that are separately described may instead be formed as a unitary or monolithic structure. In some instances, an element that is described as a single element may be manufactured as two or more distinct elements.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following figures illustrate selected components and/or arrangements of an implant for occluding the left atrial appendage, a system for occluding the left atrial appendage, and/or methods of using the implant and/or the system. It should be noted that in any given figure, some features may not be shown, or may be shown schematically, for simplicity. Additional details regarding some of the components of the implant and/or the system may be illustrated in other figures in greater detail. While discussed in the context of occluding the left atrial appendage, the implant and/or the system may also be used for other interventions and/or percutaneous medical procedures within a patient. Similarly, the devices and methods described herein with respect to percutaneous deployment may be used in other types of surgical procedures, as appropriate. For example, in some examples, the devices may be used in a non-percutaneous procedure. Devices and methods in accordance with the disclosure may also be adapted and configured for other uses within the anatomy.

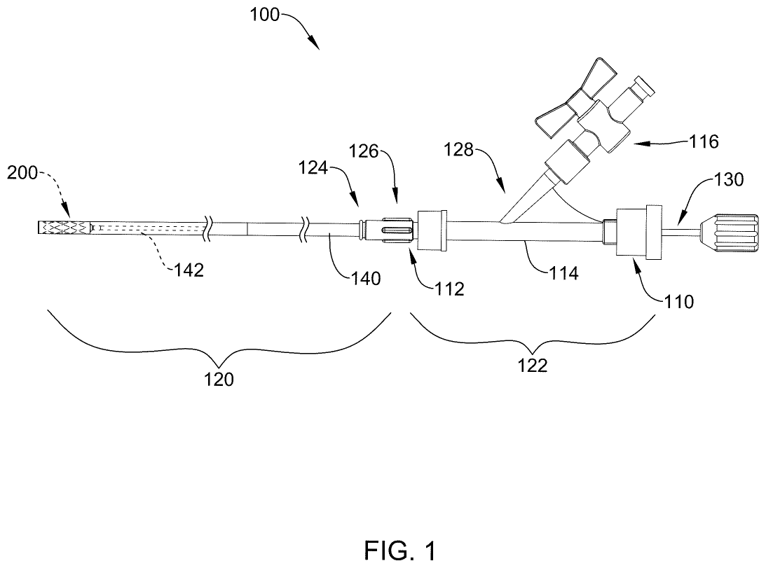

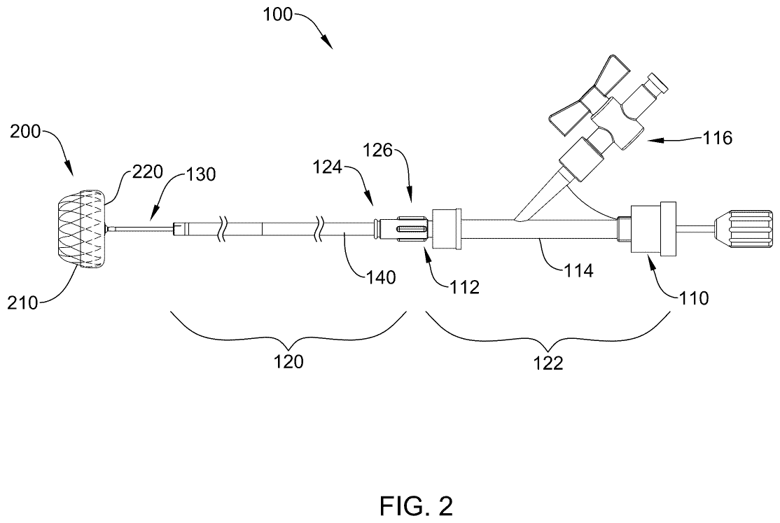

FIGS. 1 and 2 schematically illustrate selected components and/or arrangements of an occlusive implant system. It should be noted that in any given figure, some features of the occlusive implant system may not be shown, or may be shown schematically, for simplicity. Additional details regarding some of the components of the occlusive implant system may be illustrated in other figures in greater detail. The occlusive implant system may be used to deliver and/or deploy a variety of medical implants (e.g., a cardiovascular implant, an occlusive implant, etc.) to one or more locations within the anatomy, including but not limited to, in some embodiments, the heart and/or the left atrial appendage. In the interest of clarity, the following discussion refers to an occlusive implant, but other medical implants may be used and/or considered with the occlusive implant system.

The occlusive implant system may include a delivery system 100 including a delivery sheath 140 having a delivery lumen 142 extending proximally from a distal end of the delivery sheath 140. In one example, the delivery lumen 142 extends from a proximal opening to a distal opening of the delivery sheath 140. The delivery system 100 may include a proximal hub 110. In some embodiments, the delivery system may include a mid-hub 112. In some instances, the delivery system 100 may include a mid-shaft 114 extending from the proximal hub 110 to the mid-hub 112. In some instances, the delivery sheath 140 may extend distally from the mid-hub 112. Other configurations are also contemplated. In some instances, the delivery system 100 may include a side port 116. In some instances, the side port 116 may be in communication with the mid-shaft 114. Other configurations are also contemplated. In some instances, the delivery system 100 and/or the delivery lumen 142 may include a proximal segment (not shown) extending within and/or through the mid-hub 112, the mid-shaft 114, and the proximal hub 110. In some instances, the proximal segment may be in fluid communication with and/or may be an extension of the delivery lumen 142 of the delivery sheath 140. In some instances, the side port 116 may be in fluid communication with the proximal segment and/or the delivery lumen 142.

In some instances, the delivery system 100 may be considered as including a delivery sheath assembly 120 and a hub assembly 122 that is releasably engageable with the delivery sheath assembly 120. As an example, the delivery sheath assembly 120 may include the delivery sheath 140 (and the delivery lumen 142 extending through the delivery sheath 140). In some instances, the delivery sheath assembly 120 may include a proximal fitting 124 at a proximal end of the delivery sheath 140. As an example, the proximal fitting 124 may be a Luer fitting, which allows other devices (such as the hub assembly 122) to be threadedly engaged with the Luer fitting. As an example, the hub assembly 122 may include a Luer adaptor 126 that threadedly engages the proximal fitting 124. The Luer fitting allows both a mechanical connection and a fluid coupling through a lumen extending through the Luer fitting. In some instances, the lumen extending through the Luer fitting may be fluidly coupled with the delivery lumen 142

In some instances, there may be some variation as to which elements are considered as being part of the delivery sheath assembly 120 and which elements are considered as being part of the hub assembly 122. As an example, the proximal fitting 124 and the Luer adaptor 126 may be integrally formed as a single element. The combined element may be considered to be part of the delivery sheath assembly 120, for example. The combined element may be considered to be part of the hub assembly 122, for example. In some instances, the Luer adaptor 126 may be integrally formed with the hub assembly 122, as another example.

The hub assembly 122 may be considered as including the proximal hub 110, the mid-hub 112 and the mid-shaft 114 that extends between the proximal hub 110 and the mid-hub 112. The hub assembly 122 may be considered as including the side port 116 as well. In some instances, the hub assembly 122 may include a hub body 128, which as shown may include both the mid-shaft 114 and a side branch to which the side port 116 is coupled. In some instances, the side branch may include another Luer fitting to which the side port 116 may be threadedly engaged. As noted, the hub assembly 122 may be releasably secured to the delivery sheath assembly 120, such as by threadedly engaging the Luer fitting 124 (at the proximal end of the delivery sheath assembly 120) with the Luer fitting 126 (at the distal end of the hub assembly 122). In some instances, as will be discussed, there may be a desire to limit relative rotation between the delivery sheath assembly 120 and the hub assembly 122. In some instances, an undesired level of rotation between the delivery sheath assembly 120 and the hub assembly 122 may occur during initial preparations of the delivery system 100 for use, including initial flushing of the delivery system 100 with saline in order to remove any air that may be entrapped within the delivery system 100.

The occlusive implant system and/or the delivery system 100 may include a core wire 130 slidably and/or rotatably disposed within the delivery lumen 142 (and the proximal segment, where present). The occlusive implant system may include an occlusive implant 200, which may be configured for implantation within a left atrial appendage, releasably engaged with and/or releasably attached to a distal end of the core wire 130. In at least some instances, the occlusive implant 200 may be a left atrial appendage closure device. In some instances, a proximal end of the core wire 130 may extend proximally of a proximal end of the delivery sheath 140 and/or the proximal opening of the delivery lumen 142 for manual manipulation by a clinician or practitioner. In some instances, the core wire 130 may be secured relative to the proximal hub 110. As a result, rotation of the hub assembly 122 relative to the delivery sheath assembly 120 may result in rotation of the core wire 130. In some instances, rotation of the core wire 130 may start to disengage the core wire 130 with the occlusive implant 200. As will be discussed, there may be a desire to limit relative rotation between the delivery sheath assembly 120 and the hub assembly 122 in order to limit rotation of the core wire 130.

In at least some instances, the delivery sheath 140 may include and/or may be formed from a polymeric material. In some instances, the delivery sheath 140 may include and/or may be formed from a plurality of polymeric materials. In some instances, the delivery sheath may include and/or may be formed from a combination of metallic and polymeric materials. In some instances, the delivery sheath 140 may include a reinforcing element, such as a mesh, a coil, a braid, etc., formed therein, embedded therein, attached thereto, etc. along at least a portion of a length of the delivery sheath 140. Other configurations are also contemplated. Some suitable, but non-limiting, examples of materials for the occlusive implant system, the core wire 130, and/or the delivery sheath 140, etc., including but not limited to metallic materials, polymeric materials, etc., are discussed below.

The occlusive implant 200 may include an expandable framework 210 (e.g., FIG. 2) configured to shift between a delivery configuration (e.g., FIG. 1), such as when the occlusive implant 200 is disposed within the delivery lumen 142 proximate the distal opening and/or within a distal portion of the delivery lumen 142, and a deployed configuration (e.g., FIG. 2) when the occlusive implant 200 is unconstrained by the delivery sheath 140.

In some instances, the expandable framework 210 may include a plurality of interconnected struts. In some embodiments, the expandable framework 210 may be compliant or semi-compliant and may generally conform to and/or be configured to sealingly engage with the shape and/or geometry of the left atrial appendage in the deployed configuration.

In some instances, a proximal end of the expandable framework 210 may be configured to releasably attach, join, couple, engage, or otherwise connect to the distal end of the core wire 130 (e.g., FIG. 2). In some instances, the proximal end of the expandable framework 210 may include a proximal hub coupled and/or non-releasably attached thereto. In some instances, the proximal hub may be configured to and/or adapted to releasably couple with, join to, mate with, or otherwise engage a distal end of the core wire 130. Other means of releasably coupling and/or engaging the expandable framework 210 to the distal end of the core wire 130 are also contemplated.

In some instances, the occlusive implant 200 may include an occlusive element 220 (e.g., a membrane, a fabric, or a tissue element, etc.) connected to, disposed on, disposed over, disposed about, or covering at least a portion the expandable framework 210. In some instances, the occlusive element 220 may be connected to, disposed on, disposed over, disposed about, or cover at least a portion of an outer (or outwardly facing) surface of the expandable framework 210.

In some instances, the occlusive element 220 may be permeable or impermeable to blood and/or other fluids, such as water. In some instances, the occlusive element 220 may include a polymeric membrane, a metallic or polymeric mesh, a porous or semi-porous filter-like material, or other suitable construction. In some instances, the occlusive element 220 prevents thrombi (e.g., blood clots, etc.) from passing through the occlusive element 220 and out of the left atrial appendage into the blood stream. In some instances, the occlusive element 220 promotes endothelization after implantation, thereby effectively removing the target site (e.g., the left atrial appendage, etc.) from the patient's circulatory system. Some suitable, but non-limiting, examples of materials for the occlusive element 220 are discussed below.

In some instances, the expandable framework 210 and/or the plurality of interconnected struts may be integrally formed and/or cut from a unitary member. In some instances, the expandable framework 210 and/or the plurality of interconnected struts may be integrally formed and/or cut from a unitary tubular member and subsequently formed and/or heat set to a desired shape in the deployed configuration. In some instances, the expandable framework 210 and/or the plurality of interconnected struts may be integrally formed and/or cut from a unitary flat member or sheet, and then rolled or formed into a tubular structure and subsequently formed and/or heat set to the desired shape in the deployed configuration. Some exemplary means and/or methods of making and/or forming the expandable framework 210 include laser cutting, machining, punching, stamping, electro discharge machining (EDM), chemical dissolution, etc. Other means and/or methods are also contemplated.

In use, the delivery sheath 140 may be advanced and/or navigated to the left atrial appendage to deliver the occlusive implant 200 thereto. In one example, the delivery sheath 140 may be advanced and/or navigated to the left atrial appendage using and/or over a guidewire. For example, the delivery sheath 140 may be advanced to the patient's left atrium and the distal end disposed adjacent to the left atrial appendage with the occlusive implant 200 disposed therein in the delivery configuration. In some instances, the delivery sheath 140 may include steering capability. After the distal end of the delivery sheath 140 is disposed adjacent to and/or at the left atrial appendage, the core wire 130 may be advanced distally relative to the delivery sheath 140 to advance the occlusive implant 200 out of the delivery sheath 140, where the occlusive implant 200 may shift to the deployed configuration.

While not expressly illustrated, in some embodiments, the occlusive implant system may further include an access device. In some embodiments, the access device may be a bi-directional steerable catheter and/or an intravascular catheter. Examples of intravascular catheters may include, but are not limited to, balloon catheters, atherectomy catheters, device delivery catheters, drug delivery catheters, diagnostic catheters, and guide catheters.

In some instances, the access device may be advanced and/or navigated to the left atrial appendage. In one example, the access device may be advanced and/or navigated to the left atrial appendage using and/or over a guidewire. For example, the access device may be advanced to the patient's left atrium and a distal tip disposed adjacent to the left atrial appendage. In some instances, the access device may include steering capability. In some instances, the delivery system 100 may be inserted through the access device. In some instances, the length of the delivery sheath 140 may be substantially equal to the length of the access device. In some instances, the length of the delivery sheath 140 may be slightly longer than the access device. During use, the delivery sheath 140 may be advanced within the access device with the occlusive implant 200 disposed therein in the delivery configuration. After the distal end of the delivery sheath 140 is disposed adjacent to and/or at the distal end of the access device, the core wire 130 may be advanced distally relative to the delivery sheath 140 and/or the access device to advance the occlusive implant 200 out of the delivery sheath 140 and the access device, where the occlusive implant 200 may shift to the deployed configuration.

In some instances, the delivery system, the delivery sheath 140, and/or the access device may be sized in accordance with its intended use. For example, the delivery system, the delivery sheath 140, and/or the access device can have a length that is in the range of about 10 to about 150 centimeters, about 25 to about 125 centimeters, about 50 to about 100 centimeters, about 25 centimeters to about 50 centimeters, about 50 to about 75 centimeters, about 75 to about 100 centimeters, etc. Other lengths are also contemplated, including but not limited to subsets of ranges disclosed herein. It is further contemplated that the outer diameter of the delivery system, the delivery sheath 140, and/or the access device may vary based on the use or application. In some examples, the outer diameter of the delivery system, the delivery sheath 140, and/or the access device may be about 2 millimeters (mm), about 3 mm (or 9 French), about 3.5 mm, about 4 mm (or 12 French), about 4.5 mm, about 5 mm (or 15 French), about 5.33 mm, about 5.5 mm, about 5.66 mm (or 17 French), about 6 mm, about 6.5 mm, about 7 mm (or 21 French), about 8 mm, or other suitable sizes. In some instances, the outer diameter of the delivery system, the delivery sheath 140, and/or the access device may be a maximum of 5.66 mm (17 French) and is preferably smaller than 5.66 mm (17 French). Other configurations are also contemplated. In some instances, it is desirable for the outer diameter of the delivery system, the delivery sheath 140, and/or the access device to be as small as possible.

In some instances, during a procedure, it may be necessary to recapture the occlusive implant 200. For example, the initial placement of the occlusive implant 200 may be incorrect and/or inadequate. Accordingly, the delivery sheath 140 and/or the access device may be configured to permit recapture of the occlusive implant 200.

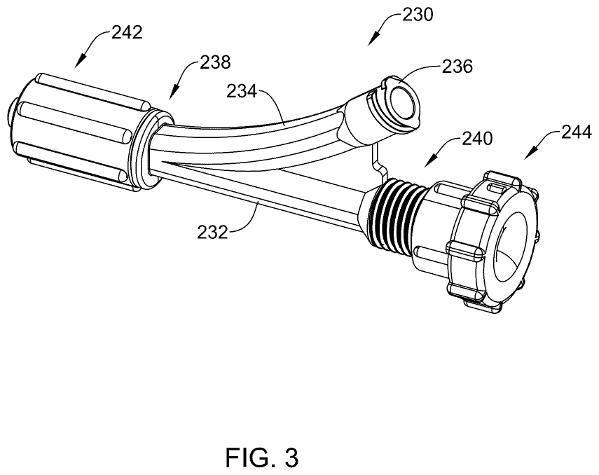

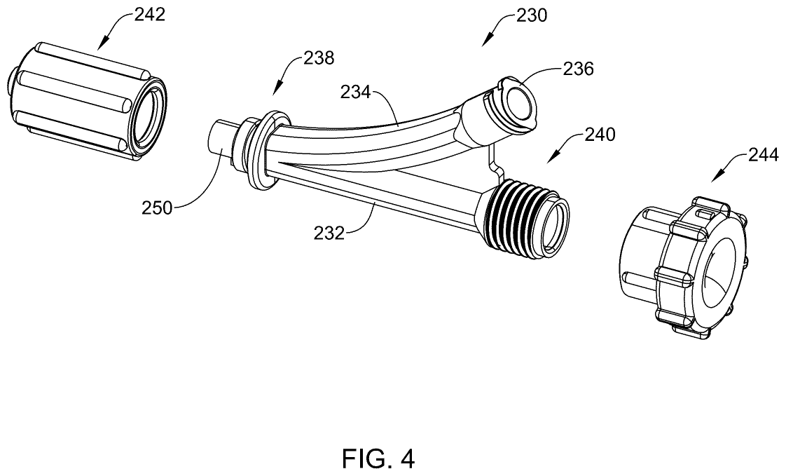

As noted above, in some instances there may be a desire to reduce or even eliminate relative rotation between the delivery sheath assembly 120 and the hub assembly 122. FIG. 3 is a perspective view of an illustrative hub assembly 230 that may be used with the delivery sheath assembly 120 (FIG. 1) in place of the hub assembly 122. FIG. 4 is an exploded perspective view of the illustrative hub assembly 130. The illustrative hub assembly 230 includes a hub body 232 that may be a molded polymeric hub body. In some instances, the hub body 232 includes a side port 234 with a Luer fitting 236. The side port 234 with the Luer fitting 236 may be considered as accommodating the side port 116 (FIG. 1). The hub body 232 includes a distal region 238 and a proximal region 240. A distal hub 242, which in some instances may be a Luer fitting, engages with the distal region 238 of the hub body 232. As will be discussed, an interaction between the distal hub 242 and the distal region 238 of the hub body 232 may provide a limitation on relative rotation between the hub assembly 230 and a delivery sheath assembly such as the delivery sheath assembly 120. A proximal hub 242, which may in some instances be an example of the proximal hub 110 (FIG. 1), may be threadedly engaged with the proximal region 240 of the hub body 232.

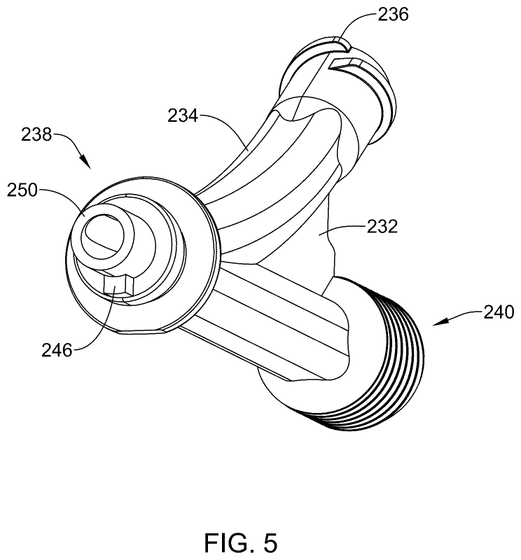

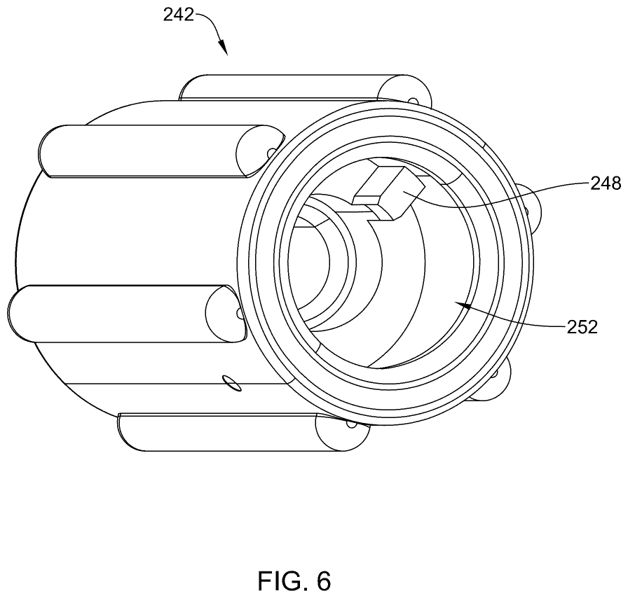

FIG. 5 is a perspective view of the hub body 232, showing the distal region 238. As can be seen, the distal region 238 of the hub body 232 includes a protrusion 246. FIG. 6, which is a perspective view of a proximal end of the distal hub 242, shows that the distal hub 242 includes a protrusion 248. In some instances, one of the protrusion 246 and the protrusion 248 may be considered as being a first protrusion and the other of the protrusion 246 and the protrusion 248 may be considered as being a second protrusion. The distal region 238 of the hub body 232 includes a reduced diameter segment 250 that extends into a corresponding annular space 252 within the distal hub 242. With the reduced diameter segment 250 extending into the corresponding annular space 252 (as shown for example in FIG. 3), it will be appreciated that relative rotation between the distal hub 242 and the distal region 238 of the hub body 232 will be limited by the protrusion 246 and the protrusion 248 running into each other. The protrusion 246 and the protrusion 248 will together limit relative rotation between the distal hub 242 and the distal region 238 of the hub body 232 will be limited to less than 360 degrees. As an example, depending on the relative dimensions of the protrusion 246 and the protrusion 248, relative rotation between the distal hub 242 and the distal region 238 of the hub body 232 may be limited to about 330 degrees.

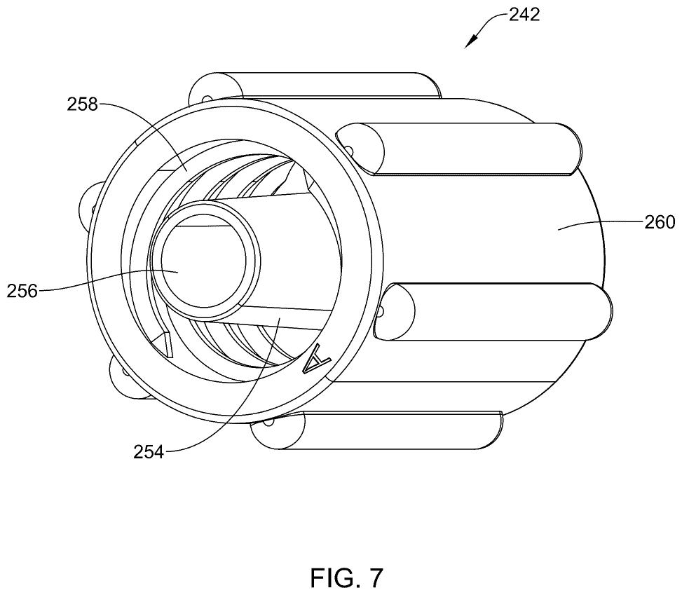

FIG. 7 is a perspective view of a distal end of the distal hub 242. As can be seen, the distal hub 242 may be considered as being a Luer adaptor or Luer nut. The distal hub 242 includes a tapered center extension 254 defining a lumen 256 extending therethrough. The distal hub 242 includes a threaded surface 258 that is adapted to threadedly engage a threaded portion of a corresponding Luer fitting (such as the Luer fitting 126 shown in FIG. 1). In some instances, the distal hub 242 includes a knurled outer surface 260 to facilitate manipulation of the distal hub 242.

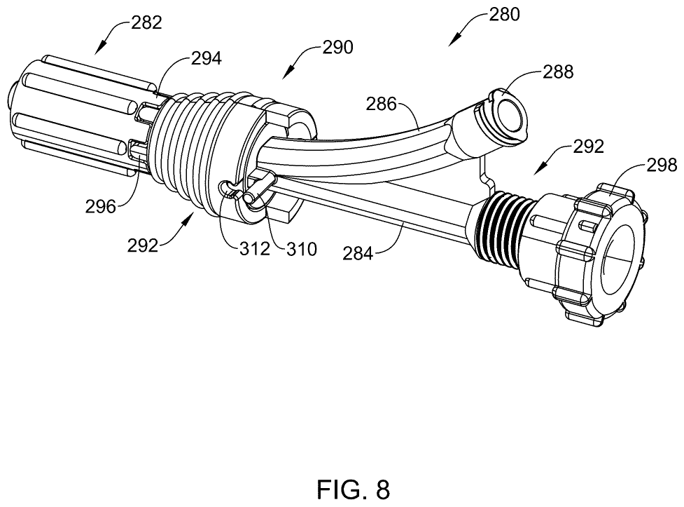

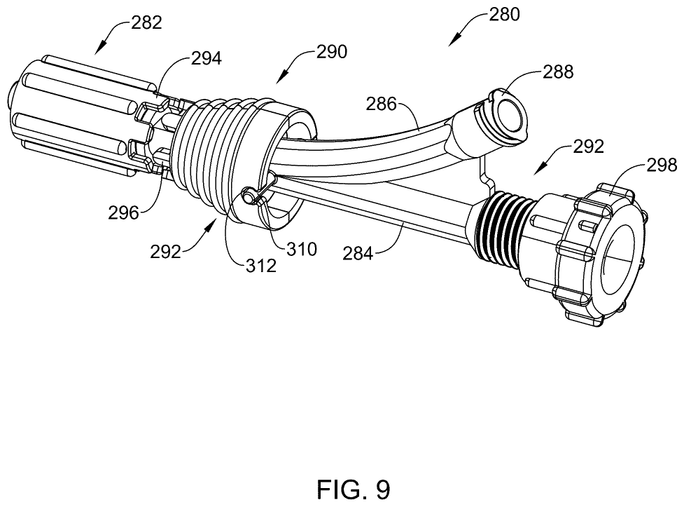

FIG. 8 is a perspective view of an illustrative hub assembly 280 that may be used with the delivery sheath assembly 120 (FIG. 1) in place of the hub assembly 122. In FIG. 8, the illustrative hub assembly 280 is shown in an engaged position, in which the hub assembly 280 is locked against rotation relative to a distal hub 282, and hence locked against rotation relative to a delivery sheath assembly to which the hub assembly 280 may be attached. In FIG. 9, the hub assembly 280 is shown in a disengaged position, in which the hub assembly 280 is not locked against rotation relative to the distal hub 282, and hence is not locked against rotation relative to a delivery sheath assembly. The hub assembly 280 includes a hub body 284 that may be a molded polymeric hub body. In some instances, the hub body 284 includes a side port 286 with a Luer fitting 288. The side port 286 with the Luer fitting 288 may be considered as accommodating the side port 116 (FIG. 1). The hub body 284 includes a distal region 290 and a proximal region 292.

A locking member 292 is disposed over the distal region 290 of the hub body 284. The locking member 292 is movable between an engaged position, in which the hub assembly 280 is locked against rotation relative to a distal hub 282, and hence locked against rotation relative to a delivery sheath assembly to which the hub assembly 280 may be attached, and a disengaged position, in which the hub assembly 280 is not locked against rotation relative to the distal hub 282, and hence is not locked against rotation relative to a delivery sheath assembly. In some instances, the distal hub 282 may be considered as including a proximal engagement feature 294 that is located along a proximal end of the distal hub 282 and the locking member 292 may be considered as including a distal engagement feature 296 that is located along a distal end of the locking member 292. A proximal hub 298, which may be considered as being an example of the proximal hub 110 (FIG. 1), is threadedly engaged with the proximal region 292 of the hub body 284.

In comparing FIG. 8 with FIG. 9, it can be seen in FIG. 8 (engaged position), the proximal engagement feature 292 and the distal engagement feature 294 have engaged each other, with each tab on one of the proximal engagement feature 292 and the distal engagement feature 294 fitting into a corresponding slot on the other of the proximal engagement feature 292 and the distal engagement feature 294. It will be appreciated that this prevents relative rotation between the distal hub 282 and the locking member 292. Conversely, in FIG. 9 (disengaged position), the proximal engagement feature 292 and the distal engagement feature 294 are not engaged with each, and relative rotation between the distal hub 282 and the locking member 292 is permitted.

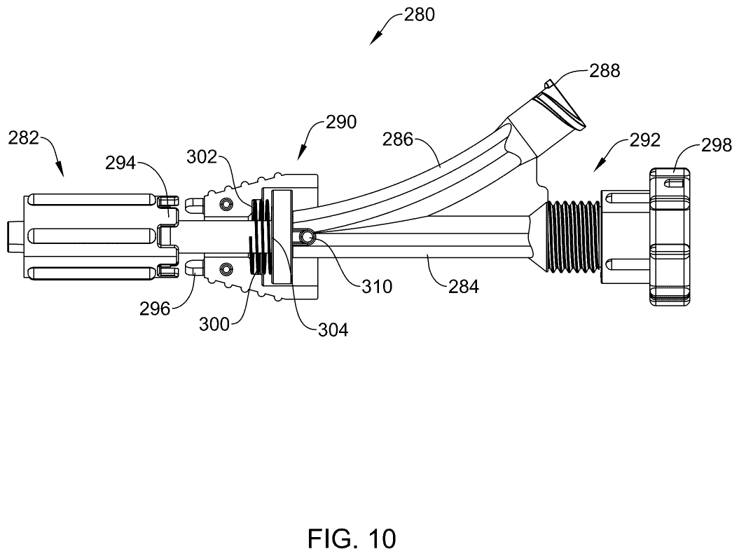

In some instances, the locking member 292 may be biased into the engaged position, and can be moved out of the engaged position against a biasing force that biases the locking member 292 into the engaged position. In FIG. 10, half of the locking member 292 has been removed to show further details. In some instances, as shown, the locking member 292 may be formed as two molded halves, that can be assembled about the distal end 290 of the hub body 284. In some instances, a spring 300 may be used to provide the biasing force. As an example, the spring 300 may be positioned between a bearing surface 302 that provides an interior stop and a backing plate 304 that is molded into the hub body 284.

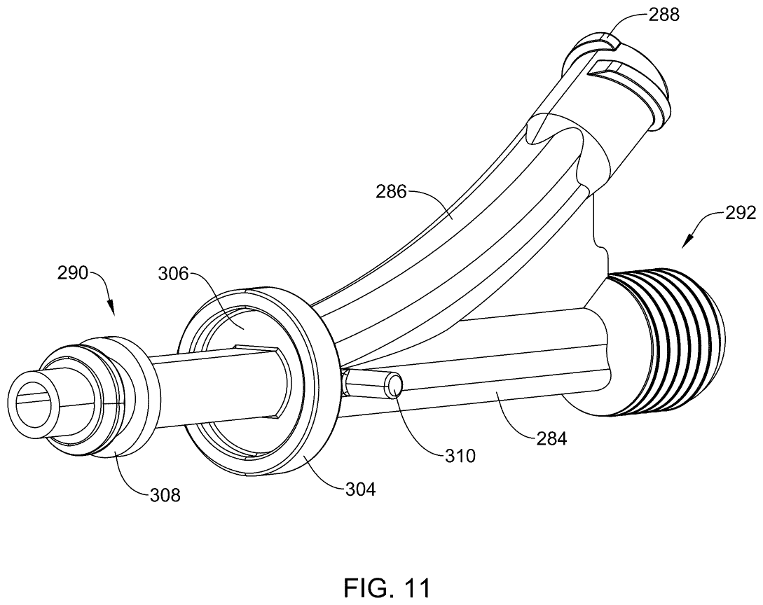

FIG. 11 is a perspective view of the hub body 284, showing the backing plate 304. The backing plate 304 may be integrally molded into the hub body 284. In some instances, as shown, the backing plate 304 includes an annular recessed portion 306 that helps to locate the spring 300. FIG. 11 also shows that the hub body 284 includes an annular flange 308 that is adapted to fit into a corresponding annular groove formed within the distal hub 282 to axially lock the distal hub 282 to the hub body 284.

The hub body 284 also includes a snap lock member 310 that may be integrally molded as part of the hub body 284. While a single snap lock member 310 is shown, it will be appreciated that the hub body 284 may include two snap lock members 310, each positioned about 180 degrees apart circumferentially. In some instances, the hub body 284 may include three or more snap lock members 310. The snap lock member(s) 310 is adapted to releasably snap into a corresponding locking feature 312 formed within the locking member 292 when the hub assembly 280 is in the disengaged position. In comparing FIG. 8 and FIG. 9, in FIG. 8 (engaged) the snap lock member 310 is spaced apart from the locking feature 312. In FIG. 9 (disengaged) the snap lock member 310 is positioned within the locking feature 312. This holds the locking member 292 in the disengaged position in order to allow relative rotation between the distal hub 282 and the locking member 290.

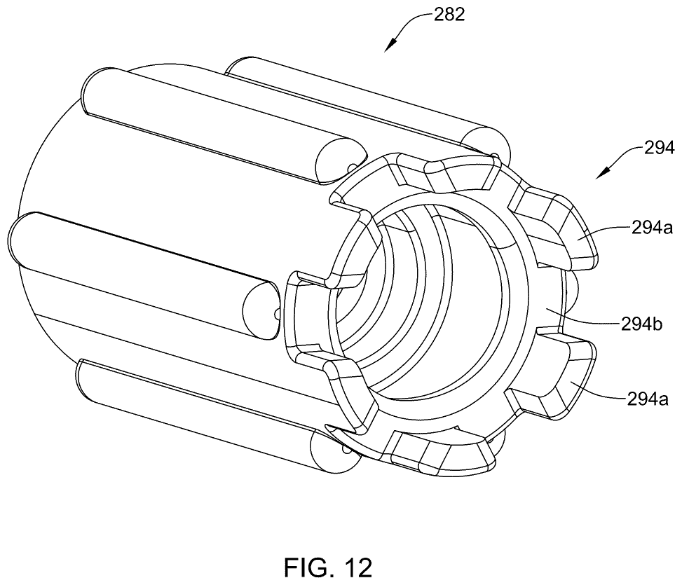

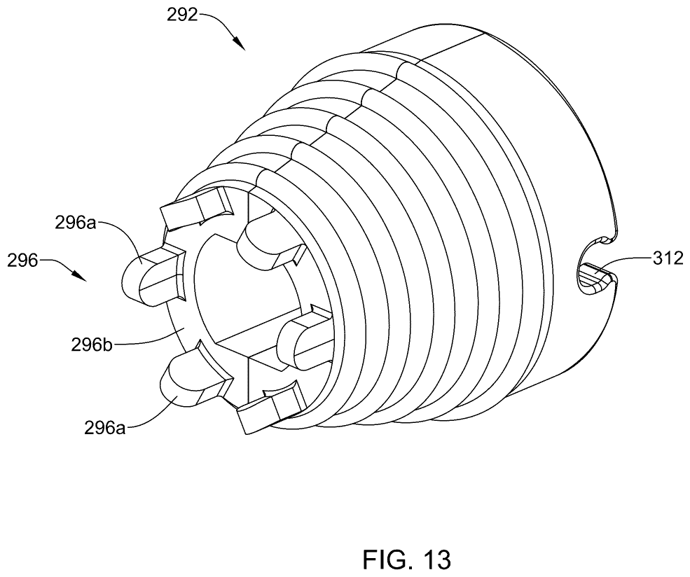

FIG. 12 is a perspective view of the proximal end of the distal hub 282, providing a better view of the proximal engagement feature 294. As can be seen, the proximal engagement feature 294 includes a number of spaced apart tabs 294a that stand proud of a surface 294b. FIG. 13 is a perspective view of the distal end of the locking member 292, providing a better view of the distal engagement feature 296. The distal engagement feature 296 includes a number of spaced apart tabs 296a that stand proud of a surface 296b. Each tab 296a fits into the space between two adjoining tabs 294a. Each tab 294a fits into the space between two adjoining tabs 296a.

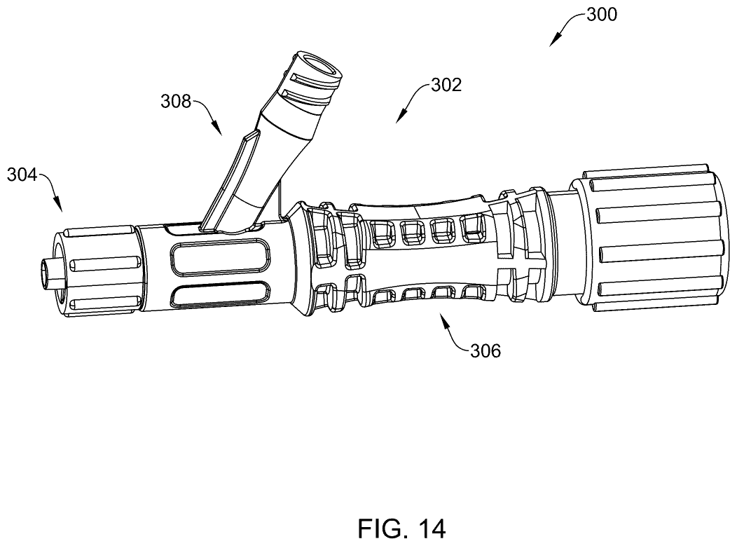

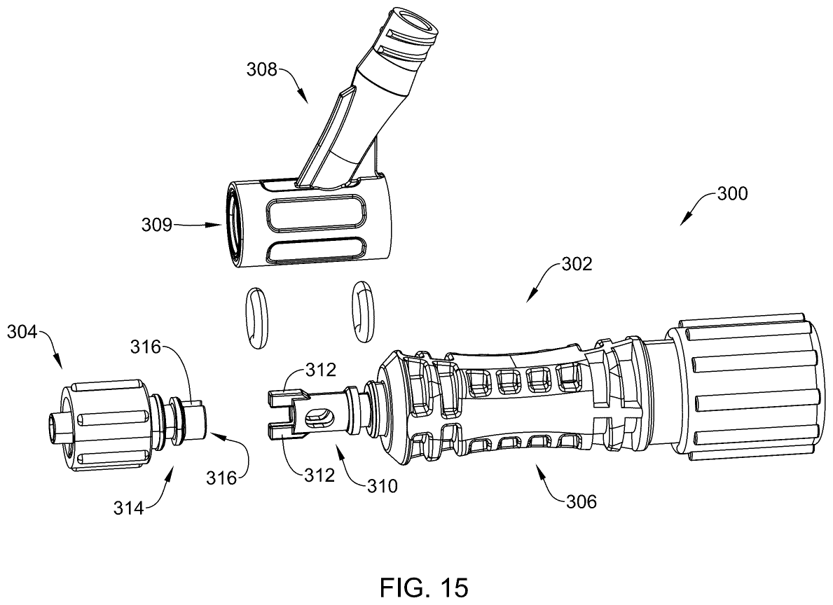

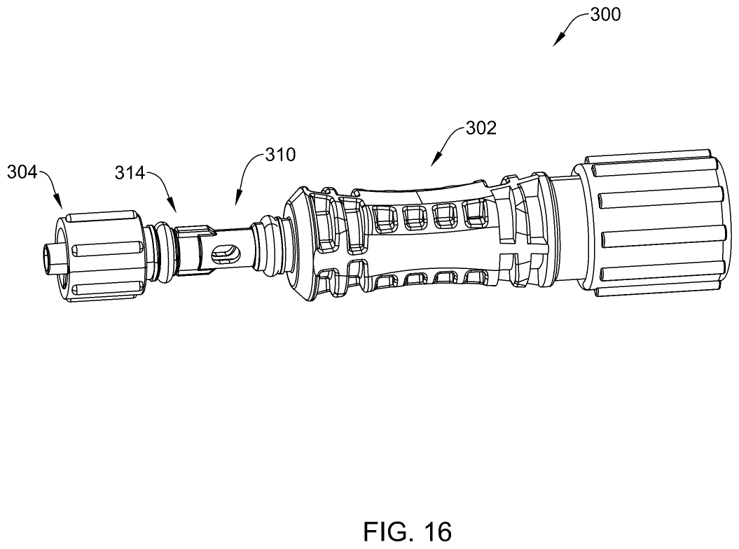

FIG. 14 is a perspective view of an illustrative hub assembly 300 that may be used with the delivery sheath assembly 120 (FIG. 1) in place of the hub assembly 122. FIG. 15 is an exploded perspective view of the illustrative hub 300. The illustrative hub 300 includes a hub body 302 and a Luer adaptor 304. In some instances, as seen for example in FIG. 15, the hub body 302 may include a main hub body section 306 and a side port body section 308. The side port body section 308 defines a lumen 309 that extends through the side port body section 308. In some instances, the side port body section 308 may be able to rotate relative to the main hub body section 306 and the Luer adaptor 304, but rotation is not permitted between the Luer adaptor 304 and the main hub body section 306. FIG. 16 shows the hub assembly 300 with the side port body section 308 removed.

The main hub body section 306 includes a narrowed distal region 310 that extends distally from the main hub body section 306. The narrowed distal region 310 includes one or more longitudinally extending tabs 312. The Luer adaptor 304 includes a narrowed proximal region 314 that extends proximally from the Luer adaptor 304. The narrowed proximal region 314 includes one or more longitudinally extending slots 316 that are complementary to the one or more longitudinally extending tabs 312. When the narrowed distal region 310 and the narrowed proximal region 314 are positioned as shown for example in FIG. 16, the one or more longitudinally extending tabs 312 engage into the one or more longitudinally extending slots 314 and rotation between the main hub body section 306 and the Luer adaptor 304 is prevented. It will be appreciated that the narrowed distal region 310 and the narrowed proximal region 314 extend towards each other and engage each other within the lumen 309 formed by the side port body section 318.

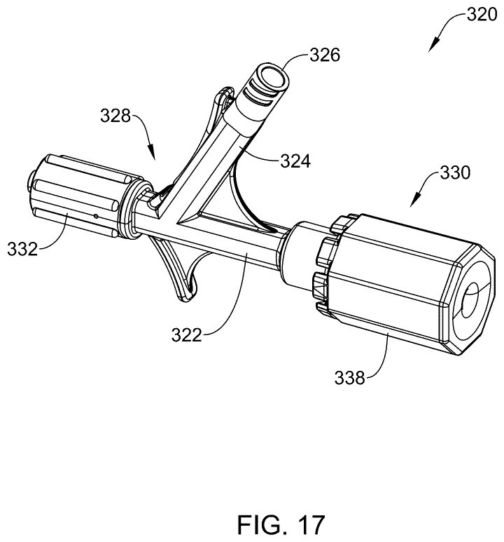

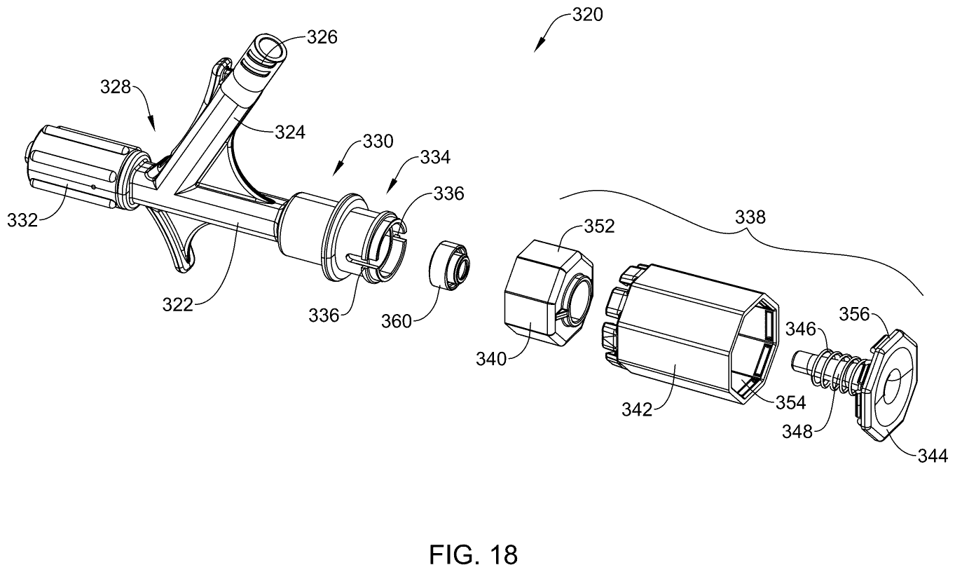

FIG. 17 is a perspective view of an illustrative hub assembly 320 that may be used with the delivery sheath assembly 120 (FIG. 1) in place of the hub assembly 122. FIG. 18 is an exploded perspective view of the illustrative hub assembly 320. The illustrative hub assembly 320 includes a hub body 322 that includes a side port branch 324 having a Luer fitting 326. The hub body 322 includes a distal region 328 and a proximal region 330. A Luer adaptor 332 is secured relative to the distal region 328. The proximal region 330 includes an annular extension 334 including a pair of circumferentially-spaced slots 336. The hub assembly 320 includes a proximal hub assembly 338 that can be caused to engage the circumferentially-spaced slots 336 in order to prevent relative rotation between the hub body 322 and the proximal hub assembly 338.

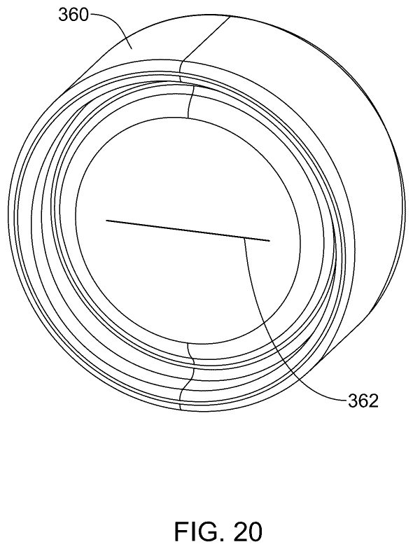

The proximal hub assembly 338 includes several components, including an engagement member 340 that, as will be discussed, is adapted to releasably engage the circumferentially-spaced slots 336 within the annular extension 334. An outer hub 342 and an inner hub 344 are also part of the proximal hub assembly 338. A spring 346 extends around a distally-extending protrusion 348 that is part of the inner hub 344. A seal 360 is disposed within the annular extension 334 and seals against a distal end of the distally-extending protrusion 348 that is part of the inner hub 344. Inclusion of the seal 360 helps to allow the core wire 130 (FIG. 1) to translate freely relative to the hub assembly 320 without leakage. A proximal side of the seal 360 is adapted to seal against an interior of the engagement member 340. With brief reference to FIG. 20, the seal 360 includes an opening 362 that allows the core wire 130 to pass through. While the opening 362 is shown as a linear slit, in some instances the opening 362 may take other forms, such as an X-shape, for example. The seal 360 may be formed of a resilient polymer that allows deformation of the opening 362 to accommodate the core wire 130 while sealing around the core wire 130.

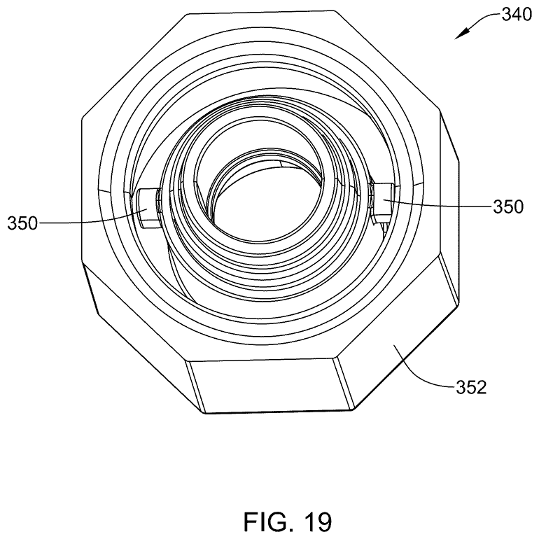

FIG. 19 is a perspective view of the distal end of the engagement member 340. As can be seen, the engagement member 340 includes a pair of tabs 350 that are adapted to engage the circumferentially-spaced slots 336 within the annular extension 334. In some instances, the spring 346 biases the proximal hub assembly 338 into a disengaged position in which the tabs 350 do not engage the circumferentially-spaced slots 336 within the annular extension 334, and thus relative rotation is permitted. By pushing the proximal hub assembly 338 distally, against the biasing force of the spring 346, the tabs 350 are able to engage the circumferentially-spaced slots 336 within the annular extension 334 and relative rotation is prevented.

In some instances, the engagement member 340 may have an outer surface 352 that is not rounded, but rather has a number of flattened sides. As an example, the outer surface 352 may have a total of eight flattened sides, but this is not required. In some instances, the outer hub 342 may have a complementary inner surface 354 with similar flattened sides in order to prevent relative rotation between the engagement member 340 and the outer hub 342. Similarly, the inner hub 344 may have an outer periphery 356 with similar flattened sides.

The materials that can be used for the various components of the occlusive implant system (and/or other elements disclosed herein) and the various components thereof disclosed herein may include those commonly associated with medical devices and/or systems. For simplicity purposes, the following discussion refers to the system. However, this is not intended to limit the devices and methods described herein, as the discussion may be applied to other elements, members, components, or devices disclosed herein, such as, but not limited to, the delivery system, the delivery sheath, the occlusive implant, the core wire, the expandable framework, the occlusive element, etc. and/or elements or components thereof.

In some instances, the system and/or components thereof may be made from a metal, metal alloy, polymer (some examples of which are disclosed below), a metal-polymer composite, ceramics, combinations thereof, and the like, or other suitable material.

Some examples of suitable metals and metal alloys include stainless steel, such as 304 and/or 316 stainless steel and/or variations thereof; mild steel; nickel alloys such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276R, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; combinations thereof; and the like; or any other suitable material.

In some instances, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof, may also be made from a material that the MRI machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-N® and the like), nitinol, and the like, and others.

In some instances, the system and/or other elements disclosed herein may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA, for example PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, polyurethane silicone copolymers (for example, Elast-Eon® or ChronoSil®), ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some instances, the system and/or other elements disclosed herein may include a fabric material disposed over or within the structure. The fabric material may be composed of a biocompatible material, such a polymeric material or biomaterial, adapted to promote tissue ingrowth. In some instances, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some instances, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, tantalum, or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some instances, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps, without exceeding the scope of the disclosure. This may include, to the extent that it is appropriate, the use of any of the features of one example being used in other examples. The disclosure's scope is, of course, defined in the language in which the appended claims are expressed.

What is claimed is:

1. A delivery system for an occlusive implant, the delivery system comprising:
a delivery sheath assembly comprising:
a delivery sheath extending from a proximal end to a distal end, the delivery sheath defining a delivery lumen extending therethrough, the delivery lumen adapted to accommodate an occlusive implant releasably secured to a core wire; and
a proximal fitting secured to the proximal end of the delivery sheath;
a hub assembly adapted to be rotatably coupled with the delivery sheath assembly, the hub assembly comprising:
a hub body;
a distal hub secured relative to the hub body and adapted to engage the proximal fitting of the delivery sheath assembly;
a proximal hub secured to the hub body, the proximal hub adapted to accommodate the core wire extending through the proximal hub; and
a side port coupled to hub body and adapted for flushing an interior of the delivery system;
wherein the delivery system includes one or more features that are adapted to limit relative rotation between the delivery sheath assembly and the hub assembly when the core wire is rotationally locked to the proximal hub.

2. The delivery system of claim 1, wherein the proximal fitting comprises a Luer fitting.

3. The delivery system of claim 2, wherein the proximal fitting and/or the distal hub include one or more features that are adapted to limit relative rotation between the delivery sheath assembly and the hub assembly.

4. The delivery system of claim 3, wherein the distal hub comprises a Luer adaptor.

5. The delivery system of claim 4, wherein:
the Luer adaptor includes a first protruding feature; and
a distal region of the hub body includes a second protruding feature adapted to engage the first protruding feature as a result of relative rotation between the Luer adaptor and the hub body;
where engagement between the first protruding feature and the second protruding feature limits relative rotation between the Luer adaptor and the hub body to less than 360 degrees.

6. The delivery system of claim 5, wherein engagement between the first protruding feature and the second protruding feature limits relative rotation between the Luer adaptor and the hub body to about 330 degrees.

7. The delivery system of claim 4, wherein the hub assembly further comprises:
a locking member moveable between an engaged position providing a rotation lock between the delivery sheath assembly and the hub assembly and a disengaged position permitting rotation between the delivery sheath assembly and the hub assembly; and
a spring that extends between an interior stop within the locking member and a backing plate molded into the hub body, the spring biasing the stopper into the engaged position.

8. The delivery system of claim 7, wherein:
the Luer adaptor comprises a proximal engagement feature; and
the locking member comprises a distal engagement feature that is complementary to the proximal engagement feature;
wherein:
the distal engagement feature engaging the proximal engagement feature when the locking member is in the engaged position; and
the distal engagement feature does not engage the proximal engagement feature when the locking member is in the disengaged position.

9. The delivery system of claim 7, wherein:
the hub body further comprises a snap lock member extending radially outwardly from the hub body; and
the locking member is adapted to releasably engage the snap lock member when the stopper is in the disengaged position.

10. The delivery system of claim 4, wherein:
the Luer adaptor comprises a narrowed proximal region including one or more longitudinally extending slots; and
the hub body comprises:
a main hub body section including a narrowed distal region, the narrowed distal region including one or more longitudinally extending tabs complementary to the one or more longitudinally extending slots;
a side port body section defining a longitudinal lumen extending therethrough.

11. The delivery system of claim 10, wherein:
the narrowed proximal region extends proximally into the longitudinal lumen;
the narrowed distal region extends distally into the longitudinal lumen such that the one or more longitudinally extending tabs engage the one or more longitudinally extending slots to prevent relative rotation between the main hub body section and the Luer adaptor; and
the side port body section is allowed to rotate relative to the main hub body section and the Luer adaptor.

12. A delivery system for an occlusive implant, the delivery system comprising:
a delivery sheath defining a delivery lumen extending therethrough that is adapted to accommodate an occlusive implant releasably secured to a core wire;
a Luer fitting secured to a proximal end of the delivery sheath;
a hub body;
a Luer adaptor secured relative to the hub body and adapted to engage the Luer fitting; and
a proximal hub secured to the hub body, the proximal hub adapted to accommodate the core wire extending through the proximal hub; and
wherein the Luer adaptor and/or the distal hub includes one or more features that are adapted to limit relative rotation between the Luer adaptor and the Luer fitting.

13. The delivery system of claim 12, wherein:
the Luer adaptor includes a first protruding feature; and
a distal region of the hub body includes a second protruding feature adapted to engage the first protruding feature as a result of relative rotation between the Luer adaptor and the hub body;
where engagement between the first protruding feature and the second protruding feature limits relative rotation between the Luer adaptor and the hub body to less than 360 degrees.

14. The delivery system of claim 13, wherein engagement between the first protruding feature and the second protruding feature limits relative rotation between the Luer adaptor and the hub body to about 330 degrees.

15. The delivery system of claim 12, further comprising:
a locking member moveable between an engaged position providing a rotation lock between the Luer fitting and the Luer adaptor and a disengaged position permitting rotation between the Luer fitting and the Luer adaptor; and
a spring that extends between an interior stop within the locking member and a backing plate molded into the hub body, the spring biasing the stopper into the engaged position.

16. The delivery system of claim 15, wherein:
the Luer adaptor comprises a proximal engagement feature; and
the locking member comprises a distal engagement feature that is complementary to the proximal engagement feature;
wherein:
the distal engagement feature engaging the proximal engagement feature when the locking member is in the engaged position; and
the distal engagement feature does not engage the proximal engagement feature when the locking member is in the disengaged position.

17. The delivery system of claim 15, wherein:
the hub body further comprises a snap lock member extending radially outwardly from the hub body; and
the locking member is adapted to releasably engage the snap lock member when the stopper is in the disengaged position.

18. The delivery system of claim 12, wherein:
the Luer adaptor comprises a narrowed proximal region including one or more longitudinally extending slots; and
the hub body comprises:
a main hub body section including a narrowed distal region, the narrowed distal region including one or more longitudinally extending tabs complementary to the one or more longitudinally extending slots;
a side port body section defining a longitudinal lumen extending therethrough.

19. The delivery system of claim 18, wherein:
the narrowed proximal region extends proximally into the longitudinal lumen;
the narrowed distal region extends distally into the longitudinal lumen such that the one or more longitudinally extending tabs engage the one or more longitudinally extending slots to prevent relative rotation between the main hub body section and the Luer adaptor; and
the side port body section is allowed to rotate relative to the main hub body section and the Luer adaptor.

20. A delivery system for an occlusive implant, the delivery system comprising:
a delivery sheath defining a delivery lumen extending therethrough;
an occlusive implant releasably secured to a core wire, the occlusive implant and the core wire disposed within the delivery lumen;
a Luer fitting secured to a proximal end of the delivery sheath;
a hub body; and
a Luer adaptor secured relative to the hub body and adapted to engage the Luer fitting;
wherein the Luer adaptor and the distal hub each include one or more features that are adapted to limit relative rotation between the Luer adaptor and the Luer fitting.
